(19) United States
(12) Patent Application Publication
(10) Pub. No.: US 2026/0329969 A1
(43) Pub. Date: Oct. 22, 2026

(54) RESAMPLING AN INPUT SIGNAL TO GENERATE MULTI-RESOLUTION FEATURES

(71) Applicant: Lenovo (United States) Inc., Morrisville, NC (US)

(72) Inventors: James Ashley, Naperville, IL (US); Razvan-Andrei Stoica, Essen (DE)

(73) Assignee: Lenovo (United States) Inc., Morrisville, NC (US)

(21) Appl. No.: 19/635,661

(22) Filed: Mar. 31, 2026

(51) Int. Cl. G06F 18/2111; G16H 10/60; H04L 41/16; H04W 24/10

(52) U.S. Cl. CPC G06F 18/2111; G16H 10/60; H04L 41/16; H04W 24/10

(57) ABSTRACT

Various aspects of the present disclosure relate to resampling an input signal to generate multi-resolution features. An apparatus (e.g., a user equipment (UE) and/or a network equipment (NE)) receives an input signal and generates a set of multi-resolution features from the input signal. The apparatus resamples the set of multi-resolution features to generate a set of resampled multi-resolution features and combines the set of resampled multi-resolution features to generate an output.

### TECHNICAL FIELD

The present disclosure relates to wireless communications, and more specifically to artificial intelligence (AI) and machine learning (ML).

### BACKGROUND

A wireless communications system may include one or multiple network communication devices, which may be otherwise known as network equipment (NE), supporting wireless communications for one or multiple user communication devices, which may be otherwise known as user equipment (UE), or other suitable terminology. The wireless communications system may support wireless communications with one or multiple user communication devices by utilizing resources of the wireless communication system (e.g., time resources (e.g., symbols, slots, subframes, frames, or the like) or frequency resources (e.g., subcarriers, carriers, or the like)). Additionally, the wireless communications system may support wireless communications across various radio access technologies including third generation (3G) radio access technology, fourth generation (4G) radio access technology, fifth generation (5G) radio access technology, among other suitable radio access technologies beyond 5G (e.g., sixth generation (6G)).

### SUMMARY

As used herein, including in the claims, an article “a” before an element is unrestricted and understood to refer to “at least one” of those elements or “one or more” of those elements. The terms “a,” “at least one,” “one or more,” and “at least one of one or more” may be interchangeable. As used herein, including in the claims, “or” as used in a list of items (e.g., a list of items prefaced by a phrase such as “at least one of” or “one or more of” or “one or both of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an example step that is described as “based on condition A” may be based on both a condition A and a condition B without departing from the scope of the present disclosure. In other words, as used herein, the phrase “based on” shall be construed in the same manner as the phrase “based at least in part on”. Further, as used herein, including in the claims, a “set” may include one or more elements.

The devices (e.g., NE, UE), processors, and methods of the present disclosure each have several innovative aspects, no single one of which is solely responsible for the desirable features disclosed herein.

A UE and/or a NE for wireless communication is described. The UE and/or the NE may be configured to, capable of, or operable to perform one or more operations as described herein. For example, the UE and/or the NE may be configured to, capable of, or operable to generate a set of multi-resolution features from an input signal; generate a set of resampled multi-resolution features based at least in part on resampling the set of multi-resolution features; and generate a processed output based at least in part on combining the set of resampled multi-resolution features.

A processor (e.g., a standalone processor chipset, or a component of a UE and/or a NE) for wireless communication is described. The processor may be configured to, capable of, or operable to perform one or more operations as described herein. For example, the processor may be configured to, capable of, or operable to generate a set of multi-resolution features from an input signal; generate a set of resampled multi-resolution features based at least in part on resampling the set of multi-resolution features; and generate a processed output based at least in part on combining the set of resampled multi-resolution features.

A method performed or performable by a UE and/or a NE for wireless communication is described. The method may include generating a set of multi-resolution features from an input signal; generating a set of resampled multi-resolution features based at least in part on resampling the set of multi-resolution features; and generating a processed output based at least in part on combining the set of resampled multi-resolution features.

In some implementations of the UE, the NE, the processor, and the method described herein, resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an electrocardiogram (ECG) signal, an electroencephalogram (EEG) signal, or a photoplethysmography (PPG) signal; a resampling function is implemented to resample the set of multi-resolution features to generate the set of resampled multi-resolution features, and wherein a derivative of the resampling function is expressed in terms of a summation of gradients of the resampling function.

A UE and/or a NE for wireless communication is described. The UE and/or the NE may be configured to, capable of, or operable to perform one or more operations as described herein. For example, the UE and/or the NE may be configured to, capable of, or operable to receive an input signal; generate a set of multi-resolution features based at least in part on processing the input signal; generate a set of resampled multi-resolution features based at least in part on resampling the set of multi-resolution features; and generate an output based at least in part on combining the set of resampled multi-resolution features.

A processor (e.g., a standalone processor chipset, or a component of a UE and/or a NE) for wireless communication is described. The processor may be configured to, capable of, or operable to perform one or more operations as described herein. For example, the processor may be configured to, capable of, or operable to receive an input signal; generate a set of multi-resolution features based at least in part on processing the input signal; generate a set of resampled multi-resolution features based at least in part on resampling the set of multi-resolution features; and generate an output based at least in part on combining the set of resampled multi-resolution features.

A method performed or performable by a UE and/or a NE for wireless communication is described. The method may include receiving an input signal; generating a set of multi-resolution features based at least in part on processing the input signal; generating a set of resampled multi-resolution features based at least in part on resampling the set of multi-resolution features; and generating an output based at least in part on combining the set of resampled multi-resolution features.

In some implementations of the UE, the NE, the processor, and the method described herein, resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an ECG signal, an EEG signal, or a PPG signal; a resampling function is implemented to resample the set of multi-resolution features to generate the set of resampled multi-resolution features, and wherein a derivative of the resampling function is expressed in terms of a summation of gradients of the resampling function.

### BRIEF DESCRIPTION OF THE DRAWINGS

FIG. 1 illustrates an example of a wireless communications system in accordance with aspects of the present disclosure.

FIGS. 2-9 illustrate example scenarios in accordance with aspects of the present disclosure.

FIG. 10 illustrates an example of a UE in accordance with aspects of the present disclosure.

FIG. 11 illustrates an example of a processor in accordance with aspects of the present disclosure.

FIG. 12 illustrates an example of an NE in accordance with aspects of the present disclosure.

FIGS. 13-18 illustrate flowcharts of methods in accordance with aspects of the present disclosure.

### DETAILED DESCRIPTION

In a wireless communications system, a UE and an NE (e.g., a base station, gNB) may support wireless communication (e.g., reception and/or transmission of wireless communication) using time-frequency resources. Many wireless communications systems and applications utilize AI/ML techniques, such as to conserve wireless resources, increase signal throughput, and/or to increase signal quality. Utilizing AI/ML techniques in wireless communications often involves different AI/ML models. For example, convolutional neural networks (CNNs) are widely used in a variety of multimedia applications. Among these applications are image enhancement, audio enhancement, image segmentation, image classification, automatic speech recognition, text-to-speech synthesis, image generation, and neural audio codecs. Many of these applications involve resampling of input data such that the output data has a modified resolution. The output resolution with respect to the input may be higher through an interpolation means, or lower through a decimation means.

Some neural audio codecs use variations on an AI/ML architecture known as a variational auto-encoder (VAE) architecture. A VAE model may compress an input audio signal down to a compressed representation, transmit or store the compressed representation for use by a decoder, and then decompress the information to reconstruct a version of the original input audio signal. A large part of the processing in such systems is therefore dedicated to successively downsampling the input signal to a low-bandwidth signal and successively upsampling the compressed representation to produce reconstructed output audio.

In CNN-based systems, the concept of receptive field (RF) is relevant to network performance with respect to processing, storage, and transmission of media (e.g., audio, images, video, etc.). The RF establishes a context over which to estimate local features of media and a large RF may provide a wider context for feature estimation. Various approaches have been proposed to increase receptive field in neural networks, including the use of large convolution kernels and deep residual network architectures. Existing encoder-decoder architectures, such as the U-Net architecture, have been applied to various signal processing tasks. In such architectures, an input signal may be progressively downsampled through an encoder to produce multi-resolution features, which are subsequently upsampled through a decoder and combined to produce an output signal.

Aspects of the present disclosure are described in the context of a wireless communications system, and include implementations that provide techniques for resampling signals using neural network architectures that incorporate multi-resolution features. In some aspects, a neural network may receive an input signal and generate a set of multi-resolution features from the input signal. The set of multi-resolution features may be resampled to produce a set of resampled multi-resolution features, and the set of resampled multi-resolution features may be combined to produce an output signal. The output signal may be stored locally on an apparatus and/or may be transmitted to a different apparatus, such as part of wireless data communication between different apparatuses.

In some cases, the resampling of the set of multi-resolution features may include up-sampling operations, down-sampling operations, or combinations thereof. The multi-resolution features may be generated at different resolutions, and the resampling operations may be performed independently across the different resolutions. The combination of the resampled multi-resolution features may produce an output signal having a resolution that differs from the resolution of the input signal.

Implementations described herein may be applicable to various types of input signals, such as audio data, image data, video data, human-based physiological data (e.g., electrocardiogram (ECG) signal, electroencephalogram (EEG) signal, photoplethysmography (PPG) signal, etc.), location data, etc. The neural network architectures described herein may process signals of different modalities and may be configured to perform resampling operations suited to the characteristics of the input signal type.

By performing the described techniques, a device in a wireless communications system can conserve device resources (e.g., data storage, processing bandwidth, transceiver resources, etc.) and increase wireless data transmission fidelity between different apparatuses in a wireless communication system.

Reference is made herein to communicating data or information, such as signaling communication resources and/or communications that are transmitted or received between devices. It is to be appreciated that other terms may be used interchangeably with communicating, such as signaling, transmitting, receiving, outputting, forwarding, retrieving, obtaining, and so forth.

Aspects of the present disclosure are described in the context of a wireless communications system. Aspects of the present disclosure are further set forth in the accompanying drawings and the description below. The description set forth herein, in connection with the accompanying drawings, describes example implementations and does not represent all the implementations that may be implemented or that are within the scope of the claims. The detailed description includes specific details for the purpose of providing an understanding of the described implementations. These implementations, however, may be practiced without these specific details. Additionally, the description set forth herein, in connection with the accompanying drawings is provided to enable a person having ordinary skill in the art to make or use the present disclosure. Various modifications to the disclosure will be apparent to a person having ordinary skill in the art, and the generic principles defined herein may be applied to other variations without departing from the scope of the present disclosure. Thus, the present disclosure is not limited to the examples and implementations described herein but is to be accorded the broadest scope consistent with the principles and novel features disclosed herein.

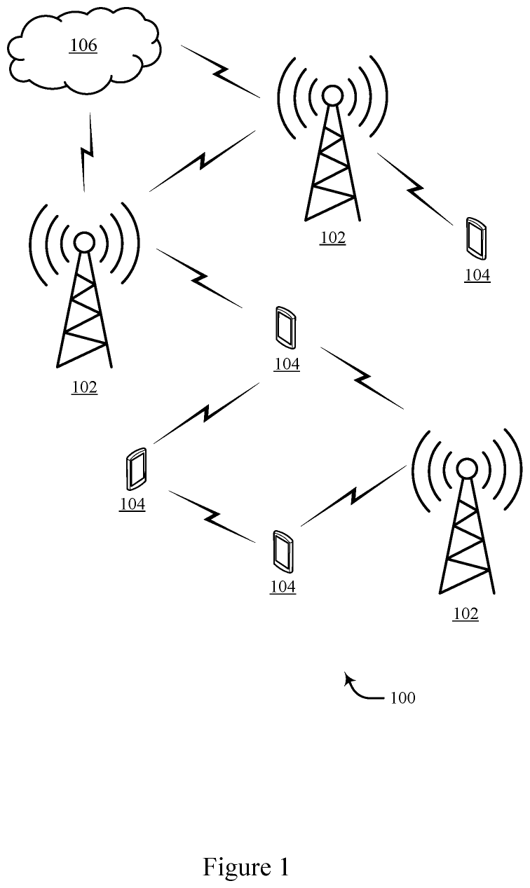

FIG. 1 illustrates an example of a wireless communications system 100 in accordance with aspects of the present disclosure. The wireless communications system 100 may include one or more NEs 102, one or more UEs 104, and a core network (CN) 106. The wireless communications system 100 may support various radio access technologies. In some implementations, the wireless communications system 100 may be a 4G network, such as an LTE network or an LTE-Advanced (LTE-A) network. In some other implementations, the wireless communications system 100 may be a NR network, such as a 5G network, a 5G-Advanced (5G-A) network, or a 5G ultrawideband (5G-UWB) network. In other implementations, the wireless communications system 100 may be a combination of a 4G network and a 5G network, or other suitable radio access technology including Institute of Electrical and Electronics Engineers (IEEE) 802.11 (Wi-Fi), IEEE 802.16 (WiMAX), IEEE 802.20. The wireless communications system 100 may support radio access technologies beyond 5G, for example, 6G. Additionally, the wireless communications system 100 may support technologies, such as time division multiple access (TDMA), frequency division multiple access (FDMA), or code division multiple access (CDMA), etc.

The one or more NEs 102 may be dispersed throughout a geographic region to form the wireless communications system 100. One or more of the NEs 102 described herein may be or include or may be referred to as a network node, a base station, an access point (AP), a network element, a network function, a network entity, a radio access network (RAN), a NodeB, an eNodeB (eNB), a next-generation NodeB (gNB), or other suitable terminology. An NE 102 and a UE 104 may communicate via a communication link, which may be a wireless or wired connection. For example, an NE 102 and a UE 104 may perform wireless communication (e.g., receive signaling, transmit signaling) over a Uu interface.

An NE 102 may provide a geographic coverage area for which the NE 102 may support services for one or more UEs 104 within the geographic coverage area. For example, an NE 102 and a UE 104 may support wireless communication of signals related to services (e.g., voice, video, packet data, messaging, broadcast, etc.) according to one or multiple radio access technologies. In some implementations, an NE 102 may be moveable, for example, a satellite associated with a non-terrestrial network (NTN). In some implementations, different geographic coverage areas associated with the same or different radio access technologies may overlap, but the different geographic coverage areas may be associated with different NE 102.

The one or more UEs 104 may be dispersed throughout a geographic region of the wireless communications system 100. A UE 104 may include or may be referred to as a remote unit, a mobile device, a wireless device, a remote device, a subscriber device, a transmitter device, a receiver device, or some other suitable terminology. In some implementations, the UE 104 may be referred to as a unit, a station, a terminal, or a client, among other examples. Additionally, or alternatively, the UE 104 may be referred to as an Internet-of-Things (IoT) device, an Internet-of-Everything (IoE) device, or machine-type communication (MTC) device, among other examples.

A UE 104 may be able to support wireless communication directly with other UEs 104 over a communication link. For example, a UE 104 may support wireless communication directly with another UE 104 over a device-to-device (D2D) communication link. In some implementations, such as vehicle-to-vehicle (V2V) deployments, vehicle-to-everything (V2X) deployments, or cellular-V2X deployments, the communication link may be referred to as a sidelink. For example, a UE 104 may support wireless communication directly with another UE 104 over a PC5 interface.

An NE 102 may support communications with the CN 106, or with another NE 102, or both. For example, an NE 102 may interface with other NE 102 or the CN 106 through one or more backhaul links (e.g., S1, N2, N6, or other network interface). In some implementations, the NE 102 may communicate with each other directly. In some other implementations, the NE 102 may communicate with each other indirectly (e.g., via the CN 106). In some implementations, one or more NEs 102 may include subcomponents, such as an access network entity, which may be an example of an access node controller (ANC). An ANC may communicate with the one or more UEs 104 through one or more other access network transmission entities, which may be referred to as a radio heads, smart radio heads, or transmission-reception points (TRPs).

The CN 106 may support user authentication, access authorization, tracking, connectivity, and other access, routing, or mobility functions. The CN 106 may be an evolved packet core (EPC), or a 5G core (5GC), which may include a control plane entity that manages access and mobility (e.g., a mobility management entity (MME), an access and mobility management function (AMF)) and a user plane entity that routes packets or interconnects to external networks (e.g., a serving gateway (S-GW), a packet data network (PDN) gateway (P-GW), or a user plane function (UPF)). In some implementations, the control plane entity may manage non-access stratum (NAS) functions, such as mobility, authentication, and bearer management (e.g., data bearers, signal bearers, etc.) for the one or more UEs 104 served by the one or more NEs 102 associated with the CN 106.

The CN 106 may communicate with a packet data network over one or more backhaul links (e.g., via an S1, N2, N6, or other network interface). The packet data network may include an application server. In some implementations, one or more UEs 104 may communicate with the application server. A UE 104 may establish a session (e.g., a protocol data unit (PDU) session, or the like) with the CN 106 via an NE 102. The CN 106 may route traffic (e.g., control information, data, and the like) between the UE 104 and the application server using the established session (e.g., the established PDU session). The PDU session may be an example of a logical connection between the UE 104 and the CN 106 (e.g., one or more network functions of the CN 106).

In the wireless communications system 100, the NEs 102 and the UEs 104 may use resources of the wireless communications system 100 (e.g., time resources (e.g., symbols, slots, subframes, frames, or the like) or frequency resources (e.g., subcarriers, carriers)) to perform various operations (e.g., wireless communications). In some implementations, the NEs 102 and the UEs 104 may support different resource structures. For example, the NEs 102 and the UEs 104 may support different frame structures. In some implementations, such as in 4G, the NEs 102 and the UEs 104 may support a single frame structure. In some other implementations, such as in 5G and among other suitable radio access technologies, the NEs 102 and the UEs 104 may support various frame structures (i.e., multiple frame structures). The NEs 102 and the UEs 104 may support various frame structures based on one or more numerologies.

One or more numerologies may be supported in the wireless communications system 100, and a numerology may include a subcarrier spacing and a cyclic prefix. A first numerology (e.g., μ=0) may be associated with a first subcarrier spacing (e.g., 15 kHz) and a normal cyclic prefix. In some implementations, the first numerology (e.g., μ=0) associated with the first subcarrier spacing (e.g., 15 kHz) may utilize one slot per subframe. A second numerology (e.g., μ=1) may be associated with a second subcarrier spacing (e.g., 30 kHz) and a normal cyclic prefix. A third numerology (e.g., μ=2) may be associated with a third subcarrier spacing (e.g., 60 kHz) and a normal cyclic prefix or an extended cyclic prefix. A fourth numerology (e.g., μ=3) may be associated with a fourth subcarrier spacing (e.g., 120 kHz) and a normal cyclic prefix. A fifth numerology (e.g., μ=4) may be associated with a fifth subcarrier spacing (e.g., 240 kHz) and a normal cyclic prefix.

A time interval of a resource (e.g., a communication resource) may be organized according to frames (also referred to as radio frames). Each frame may have a duration, for example, a 10-millisecond (ms) duration. In some implementations, each frame may include multiple subframes. For example, each frame may include 10 subframes, and each subframe may have a duration, for example, a 1 ms duration. In some implementations, each frame may have the same duration. In some implementations, each subframe of a frame may have the same duration.

Additionally, or alternatively, a time interval of a resource (e.g., a communication resource) may be organized according to slots. For example, a subframe may include a number (e.g., quantity) of slots. The number of slots in each subframe may also depend on the one or more numerologies supported in the wireless communications system 100. For instance, the first, second, third, fourth, and fifth numerologies (i.e., μ=0, μ=1, μ=2, μ=3, μ=4) associated with respective subcarrier spacings of 15 kHz, 30 kHz, 60 kHz, 120 kHz, and 240 kHz may utilize a single slot per subframe, two slots per subframe, four slots per subframe, eight slots per subframe, and 16 slots per subframe, respectively. Each slot may include a number (e.g., quantity) of symbols (e.g., OFDM symbols). In some implementations, the number (e.g., quantity) of slots for a subframe may depend on a numerology. For a normal cyclic prefix, a slot may include 14 symbols. For an extended cyclic prefix (e.g., applicable for 60 kHz subcarrier spacing), a slot may include 12 symbols. The relationship between the number of symbols per slot, the number of slots per subframe, and the number of slots per frame for a normal cyclic prefix and an extended cyclic prefix may depend on a numerology. It should be understood that reference to a first numerology (e.g., μ=0) associated with a first subcarrier spacing (e.g., 15 kHz) may be used interchangeably between subframes and slots.

In the wireless communications system 100, an electromagnetic (EM) spectrum may be split, based on frequency or wavelength, into various classes, frequency bands, frequency channels, etc. By way of example, the wireless communications system 100 may support one or multiple operating frequency bands, such as frequency range designations FR1 (410 MHz-7.125 GHz), FR2 (24.25 GHz-52.6 GHz), FR3 (7.125 GHZ-24.25 GHZ), FR4 (52.6 GHz-114.25 GHZ), FR4a or FR4-1 (52.6 GHz-71 GHz), and FR5 (114.25 GHZ-300 GHz). In some implementations, the NEs 102 and the UEs 104 may perform wireless communications over one or more of the operating frequency bands. In some implementations, FR1 may be used by the NEs 102 and the UEs 104, among other equipment or devices for cellular communications traffic (e.g., control information, data). In some implementations, FR2 may be used by the NEs 102 and the UEs 104, among other equipment or devices for short-range, high data rate capabilities.

FR1 may be associated with one or multiple numerologies (e.g., at least three numerologies). For example, FR1 may be associated with a first numerology (e.g., μ=0), which includes 15 kHz subcarrier spacing; a second numerology (e.g., μ=1), which includes 30 kHz subcarrier spacing; and a third numerology (e.g., μ=2), which includes 60 kHz subcarrier spacing. FR2 may be associated with one or multiple numerologies (e.g., at least two numerologies). For example, FR2 may be associated with a third numerology (e.g., μ=2), which includes 60 kHz subcarrier spacing; and a fourth numerology (e.g., μ=3), which includes 120 kHz subcarrier spacing.

Reference is made herein to communicating data or information, such as signaling communication resources and/or communications that are transmitted or received between devices. It is to be appreciated that other terms may be used interchangeably with communicating, such as signaling, transmitting, receiving, outputting, forwarding, retrieving, obtaining, and so forth.

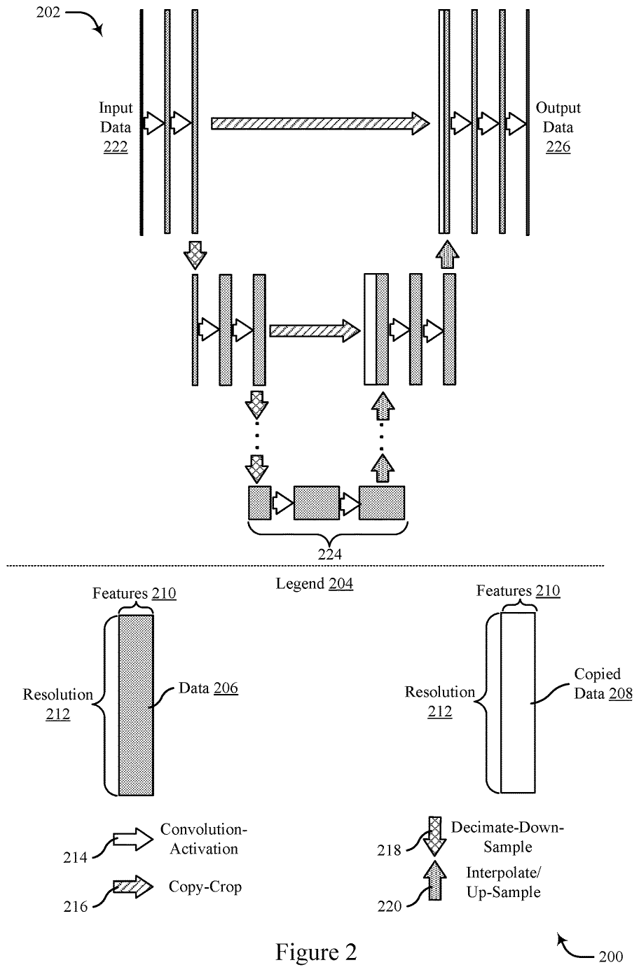

FIG. 2 illustrates an example scenario 200 in accordance with aspects of the present disclosure. The scenario 200 includes an architecture 202 and a legend 204 that explains aspects of the architecture 202. Unless otherwise indicated, features and descriptions with the legend 204 may apply to the different figures, implementations, and scenarios described herein. In implementations, the architecture 202 represents a CNN, such as a U-net architecture. As indicated by the legend 204, the architecture 202 includes data blocks 206 and copied data blocks 208. The architecture 202 can be implemented to process the data blocks 206 and the copied data blocks 208 to perform different types of data processing. The data blocks 206 and the copied data blocks 208 include different attributes including features 210 and resolution 212.

The features 210 may represent a number of different data features of data to be processed by the architecture 202. For example, where the data blocks 206 represent image data, the features 210 may represent different visual features of a digital image. In another example, where the data blocks 206 represent audio data, the features 210 may represent different audio features of digital audio. In implementations, the width of the data blocks 206 indicates a number of data features included in the data blocks 206, with wider data blocks 206 including more features than narrower data blocks 206. The resolution 212 of the data blocks 206 and the copied data blocks 208 represents an amount of detail of the data included in the data blocks 206 and the copied data blocks 208. For example, where the data blocks 206 represent image data, the resolution 212 may represent an amount of visual detail (e.g., a number of pixels and/or a pixel density) included in the data blocks 206. In another example, where the data blocks 206 include audio data, the resolution 212 may represent a bit depth and/or sample rate of the audio data included in the data blocks 206. In implementations, the height of the data blocks 206 indicates a resolution of data features included in the data blocks 206, with taller data blocks 206 representing higher resolution features than shorter data blocks 206.

The legend 204 also illustrates different operations that can be performed on the data blocks 206, including convolution-activation 214, copy-crop 216, decimate-down-sample 218, and interpolate-up-sample 220. Convolution-activation 214 may include operations such as pattern detection in data blocks 206, which features of the data blocks 206 to process in the architecture 202, and/or how to weight features of the data blocks 206 to be processed in the architecture 202. Copy-crop 216 may include operations such as copying data blocks 206 (e.g., to generate copied data blocks 208) and/or cropping data blocks 206. Decimate-down-sample 218 may include operations such as reducing the spatial and/or temporal resolution of the data blocks 206. Interpolate-up-sample 220 may include increasing the spatial and/or temporal resolution of the data blocks 206.

In the scenario 200, the architecture 202 receives input data 222. The input data 222 can include different types of data, such as image data, audio data, biometric data, location data, and/or combinations thereof. In the architecture 202, the input data 222 is progressively down-sampled via decimate-down-sample 218 to produce a set of down-sampled multi-resolution features 224. The multi-resolution features 224 are subsequently up-sampled via interpolate-up-sample 220 and combined to produce output data 226 that has a similar resolution as the input data 222. In at least some implementations, the output data 226 may be cropped via copy-crop 216. The output data 226 may be stored and/or transmitted to another apparatus. For example, the architecture 202 may be implemented on a UE 104 which can transmit the output data 226 to a different apparatus, such as an NE 102 and/or a different UE 104. Alternatively, or in addition, the architecture 202 may be implemented on a NE 102 which can transmit the output data 226 to a UE 104 and/or a different NE 102.

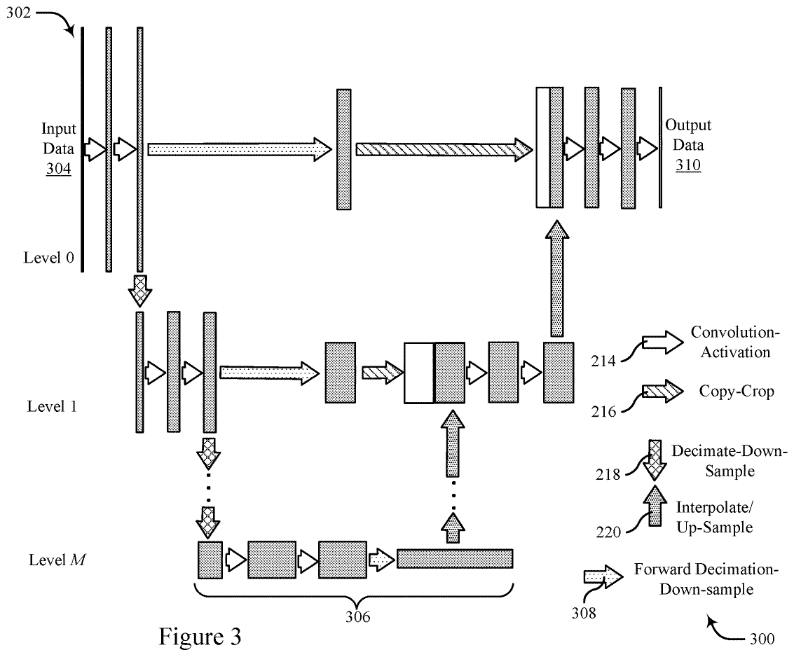

FIG. 3 illustrates an example scenario 300 in accordance with aspects of the present disclosure. The scenario 300 includes an architecture 302 in which input data 304 is progressively down-sampled as data blocks 206 at different processing levels (Level 0, Level 1, . . . . Level M) via decimate-down-sample 218 to generate a set of down-sampled multi-resolution features 306. At the different processing levels the down-sampled multi-resolution features 306 are resampled via forward decimation-down-sample 308 and combined via interpolate-up-sample 220 to generate output data 310.

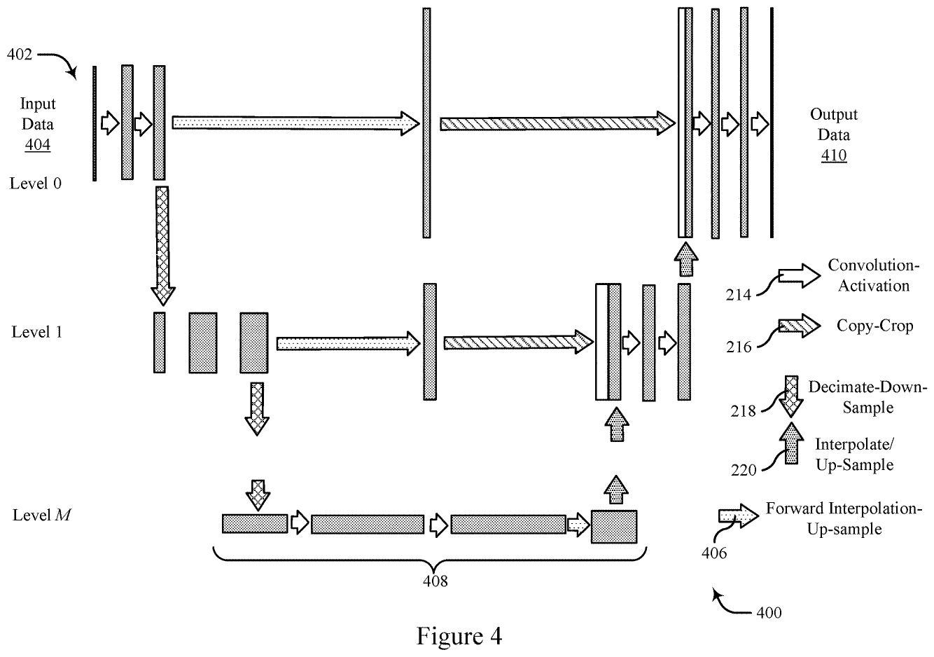

FIG. 4 illustrates an example scenario 400 in accordance with aspects of the present disclosure. The scenario 400 includes an architecture 402 in which input data 404 is progressively up-sampled as data blocks 206 at different processing levels (Level 0, Level 1, . . . . Level M) via forward interpolation up-sample 406 to generate a set of up-sampled multi-resolution features 408. The up-sampled multi-resolution features 408 are combined via interpolate-up-sample 220 to generate output data 410. In implementations (e.g., as described above), sets of down-sampled multi-resolution features may be resampled independently over the cross-section of the respective architectures. For example, there may be M+1 separate resampling operations, each at a different resolution. This provides several advantages, such as in terms of receptive field and model training.

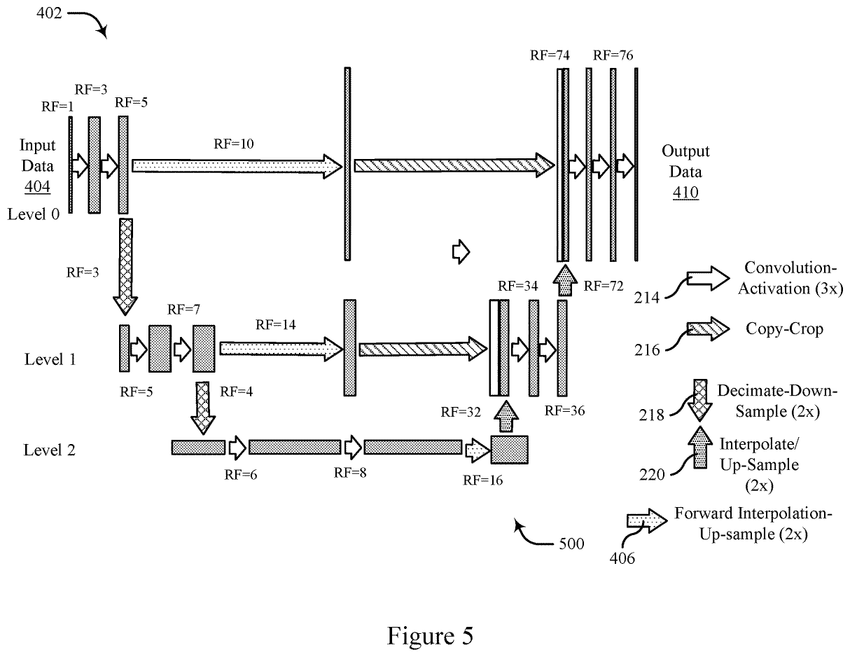

FIG. 5 illustrates an example scenario 500 in accordance with aspects of the present disclosure. The scenario 500 includes the architecture 402, such as described with reference to the scenario 400. The scenario 500 illustrates an example of how RF may be calculated over the implementation of the scenario 400. In an example, the input data 404 is of one dimension (e.g., an audio frame), and size three convolution kernels may be used for convolution-activation 214. For the convolution-activation 214, the RF grows by 2 for each pass through convolution-activation 214. For the resampling operations, a 2× increase in RF for interpolate-up-sample 220 may be used and a division by 2 for the decimate-down-sample 218, which may be common for max-pool, average pool, and strided convolutions or deconvolutions.

To perform an RF analysis on the architecture 402, a serial 3× convolutions+convolution transpose may be calculated as (1+2N)×2=76, with N stages=19 3× convolutions for RF=76. Using these assumptions, the resulting RF is calculated to be 76. This means that a single sample on the input can affect as many as 76 samples on the output. Based on an RF analysis of the scenario 500: Due to Level 0, RF=14; due to Level 1, RF=40; due to Level 2, RF=76. The scenario 500 may thus provide a total RF=76.

Furthermore, note that implementations may provide an increased RF contribution for the deeper levels of the network. The example scenario 500 illustrates RFs of 14, 40, and 76 for each Level 0, 1, and 2, respectively. Deeper levels of the network using the architecture 402 may correspond to lower frequency signals/features. This may be due to the decimation/downsampling operations having a low-pass filter effect. This property may result in the network representing a type of multi-resolution filter-bank, where the lower frequencies are represented by a larger receptive field, and the higher frequencies are represented by a smaller receptive field. This is significant because it gives the overall resampling operation more frequency domain context when compared to a “flat” CNN resampling element with a large kernel size, or a long sequence of 3× convolutions.

Implementations may facilitate improved training of a model based on the parallel nature of the resampling elements. For example, for a neural model, the partial derivative of the error E with respect to the input x can be expressed as:

$$\frac{\partial E}{\partial x} = \frac{\partial E}{\partial {f}_{net}} \frac{\partial {f}_{net}}{\partial x} , \qquad ( 1 )$$

where ∂E/∂ƒₙₑₜ is the partial derivative of the error (loss) function, and ∂ƒₙₑₜ/∂x is the partial derivative of the neural network model with respect to the input.

In at least some implementations, the second term above can be expressed as:

$$\frac{\partial {f}_{net}}{\partial x} = {\sum }_{i = 0}^{M} ( \frac{\partial {f}_{post} ( i )}{\partial {f}_{resample} ( i )} \frac{\partial {f}_{resample} ( i )}{\partial {f}_{pre} ( i )} \frac{\partial {f}_{p r e} ( i )}{\partial x} ) , \qquad ( 2 )$$

where M is the number of levels in the network, ƒₚₒₛₜ(i) is the output combining layer, ƒₚᵣₑ(i) is input decomposition layer, and ƒᵣₑₛₐₘₚₗₑ(i) is the i-th resampling layer in accordance with the current invention. In implementations, the derivative of the resampling function can be expressed in terms of a summation of the gradients (derivatives) of the resampling function ƒᵣₑₛₐₘₚₗₑ(i). One result of this property is that as the number of levels M increases, the backpropagated gradients become more and more smooth (based on the law of large numbers). This may reduce the probability that the network will converge to a poor solution (local minimum).

In the case of a serial residual network, there may only be a single instance of a resampling operation, such that the gradient may be expressed as:

$$\frac{\partial {f}_{net}}{\partial x} = \frac{\partial {f}_{net}}{\partial {f}_{resample}} \frac{\partial {f}_{resample}}{\partial {f}_{pre}} \frac{\partial {f}_{pre}}{\partial x} , \qquad ( 3 )$$

which does not share the benefit of the multi-resolution gradient sum as given in the implementations described herein.

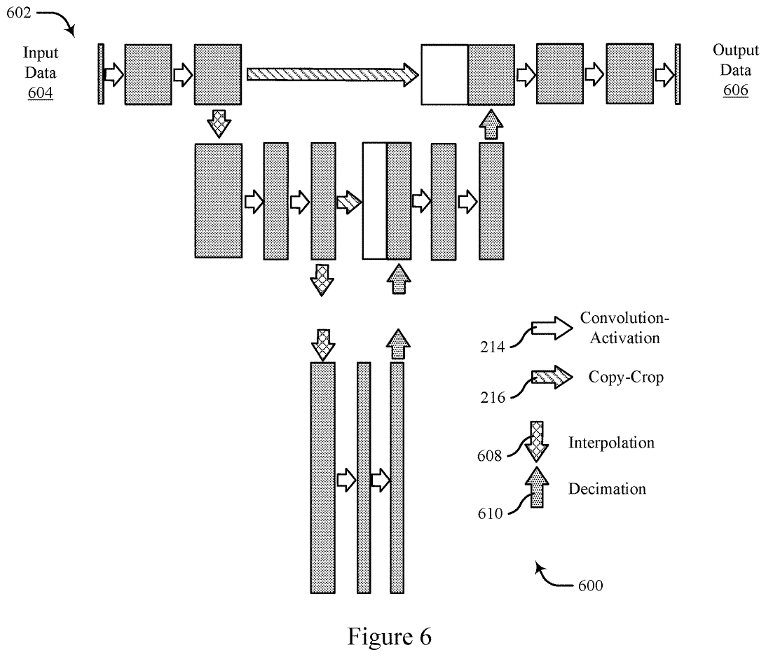

FIG. 6 illustrates an example scenario 600 in accordance with aspects of the present disclosure. The scenario 600 includes an architecture 602 in which some of the architectures described herein are modified to accommodate input that may include a large number of features. For example, multi-resolution resampling of input data 604 may be performed in an inverse manner when compared to some of the architectures described herein to generate output data 606. In implementations, the input data 604 may be successively up-sampled via interpolation 608 and accordingly down-sampled via decimation 610, and the resulting multi-resolution features are combined to form the output data 606.

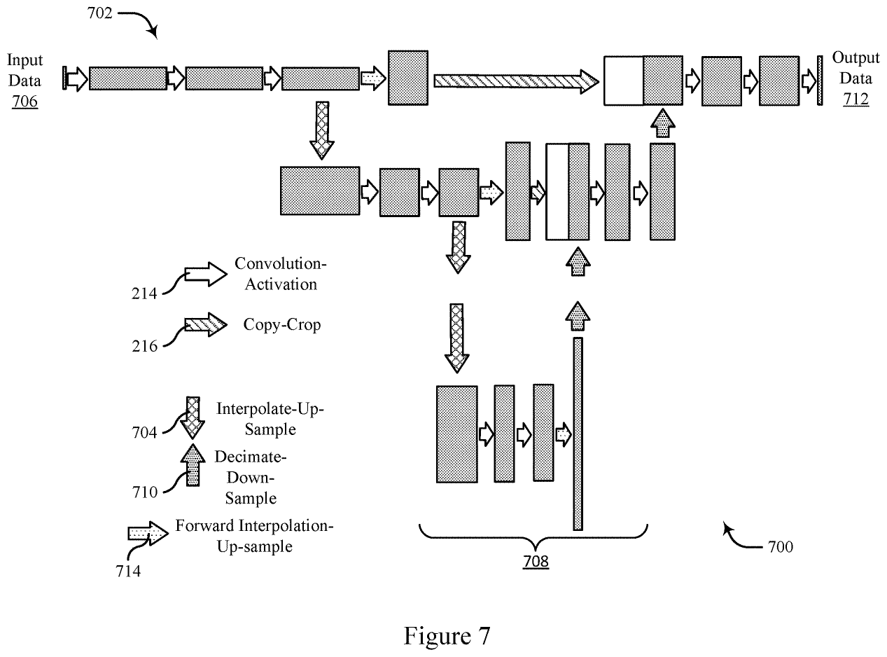

FIG. 7 illustrates an example scenario 700 in accordance with aspects of the present disclosure. The scenario 700 includes an architecture 702, which may represent an implementation of the architecture 602. In the architecture 702, cross-section resampling is performed. In the scenario 700, interpolate up-sample 704 is performed on input data 706 to generate an up-sampled set of multi-resolution features which are then resampled to produce a set of resampled up-sampled multi-resolution features 708. The resampled up-sampled multi-resolution features are combined using successive down-sampling operations 710 to produce a resampled output data 712.

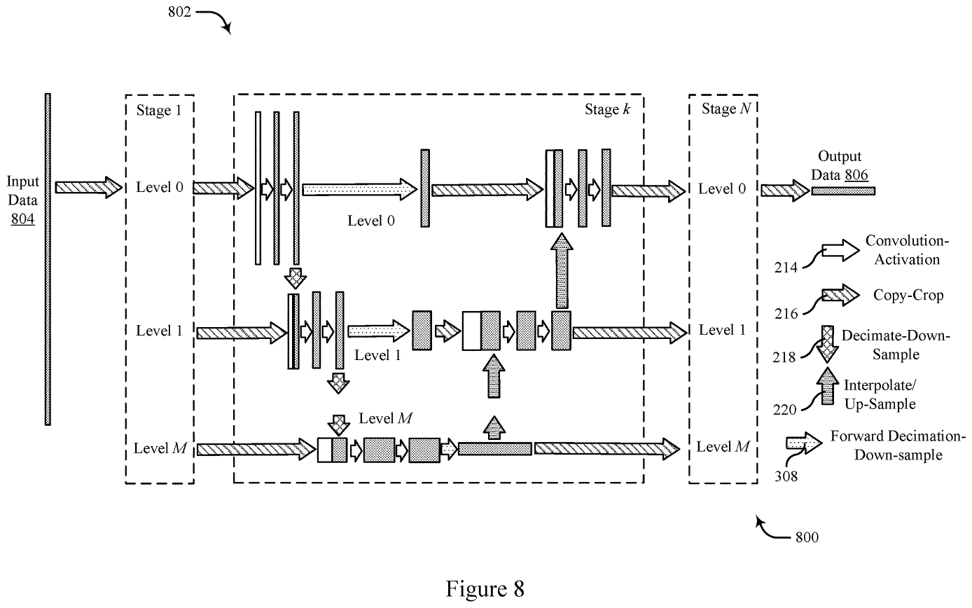

FIG. 8 illustrates an example scenario 800 in accordance with aspects of the present disclosure. The scenario 800 includes an architecture 802 which represents a combination of some of the example architectures described herein. In at least one implementation, the architecture 802 represents an encoder, e.g., a VAE. In the architecture 802, multi-resolution resampling modules may be cascaded to provide higher-order resampling tasks. In the architecture 802, the cascade involves not only the Level 0 input of input data 804 and output of output data 806, but also, one or more of the “hidden” multi-resolution features from Levels 1, . . . , M may be cascaded as well. This allows for a more comprehensive resampling system due to the distributed nature of the resampling elements. This is especially useful in the context of VAEs because of the high degree of resampling that is to take place. In addition, it may be advantageous to output “Level M” rather than Level 0 on the last stage N, due to possible redundant and/or unnecessary computations. In this case, Level 0, . . . , M−1 resampling operations may not be necessary.

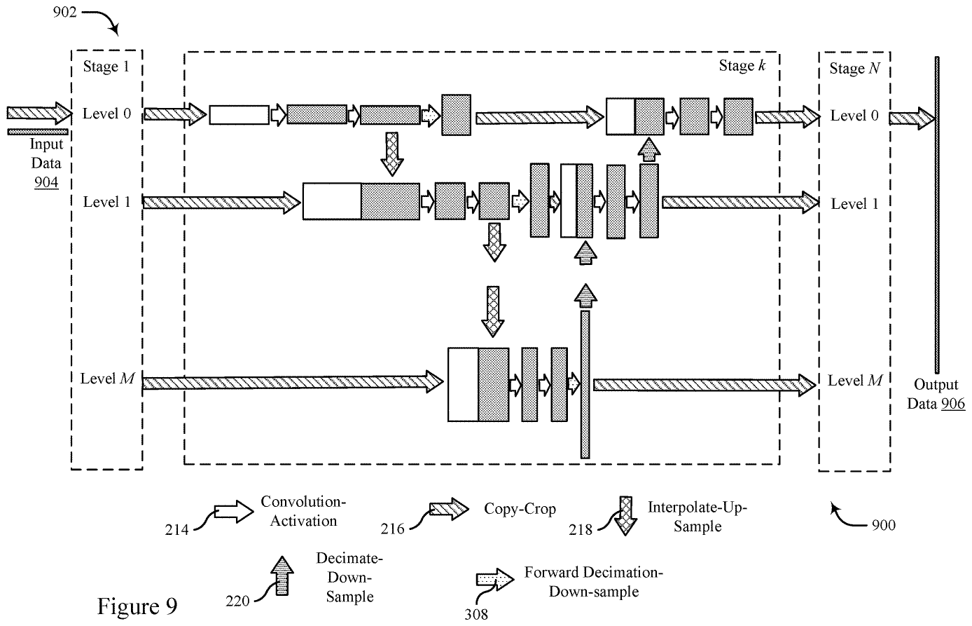

FIG. 9 illustrates an example scenario 900 in accordance with aspects of the present disclosure. The scenario 900 includes an architecture 902, which may represent a decoder version of the architecture 802. The architecture 902 may receive input data 904 (e.g., the output data 806) and decode the input data 904 to generate output data 906. The architecture 902 may be used in a cascade of operations to produce a high order set of upsampling operations. Similar to the encoder side (e.g., architecture 802), one or more of the “hidden” multi-resolution features may be cascaded as well. Similarly, the input data 904 may be inserted at Level M to avoid some redundant and/or unnecessary operations.

To illustrate the advantage of cascaded multi-resolution feature stages, Equation 2 is expanded here to show the effects of this cascade.

$$\frac{\partial {f}_{net}}{\partial x} = {\sum }_{j = 0}^{{( M + 1 )}^{N} - 1} {\prod }_{s = 1}^{N} ( \frac{\partial {f}_{post} ( s , i )}{\partial {f}_{resample} ( s , i )} \frac{\partial {f}_{resample} ( s , i )}{\partial {f}_{pre} ( s , i )} \frac{\partial {f}_{pre} ( s , i )}{\partial x} ) , \qquad ( 5 )$$

$$where i = mod ( ⌊ \frac{j}{{( M + 1 )}^{s - 1}} ⌋ , M + 1 )$$

When compared to Equation 2, the product terms over N stages show the compounding gradient effect, which is not necessarily desirable since it may amplify local minima. What is desirable, however, is the large range of values evaluated over the gradient product sums. Since there are N stages each having M+1 levels, the total number of unique gradients in the given network is (M+1)^{N}. As an example of the power of this technique, suppose we have N=4 stages, and M=4 (total of 5) resolutions per stage. Then the total number of unique gradients in the sum is 625. Now if the law of large numbers holds, this number is more than sufficient to render a favorable gradient distribution such that a model using this network is more likely to converge to a global optimum.

In addition, some architectural variations may also be considered. For example, both encoder and decoder may place the forward resampling operation at the inter-stage cross-connects. This is viewed as an equivalent configuration due to the symmetry of the network. That is, if the forward resampling operations were placed at the inter-stage connections, and the diagram of the network were flipped vertically, then the individual connections between the network elements would be very similar to that illustrated in FIGS. 8 and 9, respectively.

Furthermore, implementations may imply a certain feature dimension multiplication/division factor of 2. That is, when an input spatial/temporal dimension may be resampled by a factor of 2, the corresponding feature dimension may be resampled by a factor of ½. Also, implementations may imply a resampling factor of 2 or ½. It is anticipated that arbitrary resampling may be used, for example: 3, ⅓, 4, ¼, 5, ⅕, etc. may be possible, such as for VAE type applications.

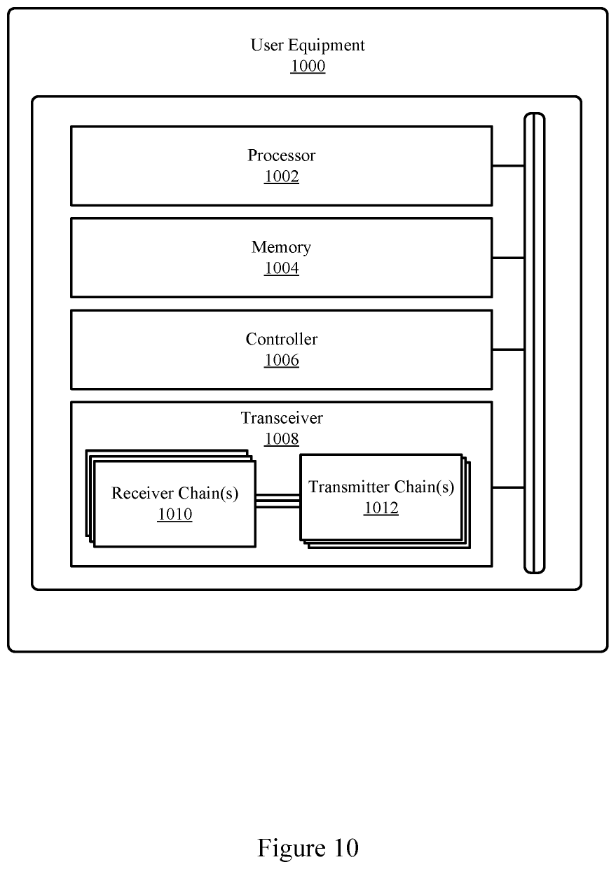

FIG. 10 illustrates an example of a UE 1000 in accordance with aspects of the present disclosure. The UE 1000 may include a processor 1002, a memory 1004, a controller 1006, and a transceiver 1008. The processor 1002, the memory 1004, the controller 1006, or the transceiver 1008, or various combinations thereof or various components thereof may be examples of means for performing various aspects of the present disclosure as described herein. These components may be coupled (e.g., operatively, communicatively, functionally, electronically, electrically) via one or more interfaces.

The processor 1002, the memory 1004, the controller 1006, or the transceiver 1008, or various combinations or components thereof may be implemented in hardware (e.g., circuitry). The hardware may include a processor, a digital signal processor (DSP), an application-specific integrated circuit (ASIC), or other programmable logic device, or any combination thereof configured as or otherwise supporting a means for performing the functions described in the present disclosure.

The processor 1002 may include an intelligent hardware device (e.g., a general-purpose processor, a DSP, a CPU, an ASIC, an FPGA, or any combination thereof). In some implementations, the processor 1002 may be configured to operate the memory 1004. In some other implementations, the memory 1004 may be integrated into the processor 1002. The processor 1002 may be configured to execute computer-readable instructions stored in the memory 1004 to cause the UE 1000 to perform various functions of the present disclosure.

The memory 1004 may include volatile or non-volatile memory. The memory 1004 may store computer-readable, computer-executable code including instructions when executed by the processor 1002 cause the UE 1000 to perform various functions described herein. The code may be stored in a non-transitory computer-readable medium such as the memory 1004 or another type of memory. Computer-readable media includes both non-transitory computer storage media and communication media including any medium that facilitates transfer of a computer program from one place to another. A non-transitory storage medium may be any available medium that may be accessed by a general-purpose or special-purpose computer.

In some implementations, the processor 1002 and the memory 1004 coupled with the processor 1002 may be configured to cause the UE 1000 to perform one or more of the functions described herein (e.g., executing, by the processor 1002, instructions stored in the memory 1004). For example, the processor 1002 may support wireless communication at the UE 1000 in accordance with examples as disclosed herein.

The UE 1000 may be configured to or operable to support a means for generating a set of multi-resolution features from an input signal; generating a set of resampled multi-resolution features based at least in part on resampling the set of multi-resolution features; and generating a processed output based at least in part on combining the set of resampled multi-resolution features.

Additionally, the UE 1000 may be configured to support any one or combination of where resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an ECG signal, an EEG, or a PPG signal; a resampling function is implemented to resample the set of multi-resolution features to generate the set of resampled multi-resolution features, and wherein a derivative of the resampling function is expressed in terms of a summation of gradients of the resampling function.

Additionally, or alternatively, the UE 1000 may support at least one memory (e.g., the memory 1004) and at least one processor (e.g., the processor 1002) coupled with the at least one memory and configured to cause the UE to generate a set of multi-resolution features from an input signal; generate a set of resampled multi-resolution features based at least in part on resampling the set of multi-resolution features; and generate a processed output based at least in part on combining the set of resampled multi-resolution features.

Additionally, the UE 1000 may be configured to support any one or combination of where resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an ECG signal, an EEG, or a PPG signal; a resampling function is implemented to resample the set of multi-resolution features to generate the set of resampled multi-resolution features, and wherein a derivative of the resampling function is expressed in terms of a summation of gradients of the resampling function.

The UE 1000 may be configured to or operable to support a means for receiving an input signal; generating a set of multi-resolution features based at least in part on processing the input signal; generating a set of resampled multi-resolution features based at least in part on resampling the set of multi-resolution features; and generating an output based at least in part on combining the set of resampled multi-resolution features.

Additionally, the UE 1000 may be configured to support any one or combination of where resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an ECG signal, an EEG, or a PPG signal; a resampling function is implemented to resample the set of multi-resolution features to generate the set of resampled multi-resolution features, and wherein a derivative of the resampling function is expressed in terms of a summation of gradients of the resampling function.

Additionally, or alternatively, the UE 1000 may support at least one memory (e.g., the memory 1004) and at least one processor (e.g., the processor 1002) coupled with the at least one memory and configured to cause the UE to receive an input signal; generate a set of multi-resolution features based at least in part on processing the input signal; generate a set of resampled multi-resolution features based at least in part on resampling the set of multi-resolution features; and generate an output based at least in part on combining the set of resampled multi-resolution features.

Additionally, the UE 1000 may be configured to support any one or combination of where resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an ECG signal, an EEG, or a PPG signal; a resampling function is implemented to resample the set of multi-resolution features to generate the set of resampled multi-resolution features, and wherein a derivative of the resampling function is expressed in terms of a summation of gradients of the resampling function.

The controller 1006 may manage input and output signals for the UE 1000. The controller 1006 may also manage peripherals not integrated into the UE 1000. In some implementations, the controller 1006 may utilize an operating system such as iOS®, ANDROID®, WINDOWS®, or other operating systems. In some implementations, the controller 1006 may be implemented as part of the processor 1002.

In some implementations, the UE 1000 may include at least one transceiver 1008. In some other implementations, the UE 1000 may have more than one transceiver 1008. The transceiver 1008 may represent a wireless transceiver. The transceiver 1008 may include one or more receiver chains 1010, one or more transmitter chains 1012, or a combination thereof.

A receiver chain 1010 may be configured to receive signals (e.g., control information, data, packets) over a wireless medium. For example, the receiver chain 1010 may include one or more antennas to receive a signal over the air or wireless medium. The receiver chain 1010 may include at least one amplifier (e.g., a low-noise amplifier (LNA)) configured to amplify the received signal. The receiver chain 1010 may include at least one demodulator configured to demodulate the received signal and obtain the transmitted data by reversing the modulation technique applied during transmission of the signal. The receiver chain 1010 may include at least one decoder for decoding the demodulated signal to receive the transmitted data.

A transmitter chain 1012 may be configured to generate and transmit signals (e.g., control information, data, packets). The transmitter chain 1012 may include at least one modulator for modulating data onto a carrier signal, preparing the signal for transmission over a wireless medium. The at least one modulator may be configured to support one or more techniques such as amplitude modulation (AM), frequency modulation (FM), or digital modulation schemes like phase-shift keying (PSK) or quadrature amplitude modulation (QAM). The transmitter chain 1012 may also include at least one power amplifier configured to amplify the modulated signal to an appropriate power level suitable for transmission over the wireless medium. The transmitter chain 1012 may also include one or more antennas for transmitting the amplified signal into the air or wireless medium.

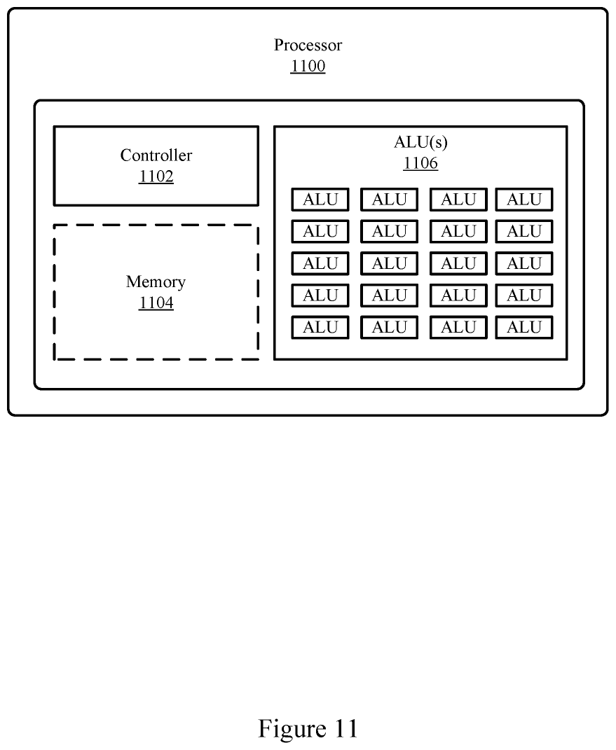

FIG. 11 illustrates an example of a processor 1100 in accordance with aspects of the present disclosure. The processor 1100 may be an example of a processor configured to perform various operations in accordance with examples as described herein. The processor 1100 may include a controller 1102 configured to perform various operations in accordance with examples as described herein. The processor 1100 may optionally include at least one memory 1104, which may be, for example, an L1/L2/L3 cache. Additionally, or alternatively, the processor 1100 may optionally include one or more arithmetic-logic units (ALUs) 1106. One or more of these components may be in electronic communication or otherwise coupled (e.g., operatively, communicatively, functionally, electronically, electrically) via one or more interfaces (e.g., buses).

The processor 1100 may be a processor chipset and include a protocol stack (e.g., a software stack) executed by the processor chipset to perform various operations (e.g., receiving, obtaining, retrieving, transmitting, outputting, forwarding, storing, determining, identifying, accessing, writing, reading) in accordance with examples as described herein. The processor chipset may include one or more cores, one or more caches (e.g., memory local to or included in the processor chipset (e.g., the processor 1100) or other memory (e.g., random access memory (RAM), read-only memory (ROM), dynamic RAM (DRAM), synchronous dynamic RAM (SDRAM), static RAM (SRAM), ferroelectric RAM (FeRAM), magnetic RAM (MRAM), resistive RAM (RRAM), flash memory, phase change memory (PCM), and others).

The controller 1102 may be configured to manage and coordinate various operations (e.g., signaling, receiving, obtaining, retrieving, transmitting, outputting, forwarding, storing, determining, identifying, accessing, writing, reading) of the processor 1100 to cause the processor 1100 to support various operations in accordance with examples as described herein. For example, the controller 1102 may operate as a control unit of the processor 1100, generating control signals that manage the operation of various components of the processor 1100. These control signals include enabling or disabling functional units, selecting data paths, initiating memory access, and coordinating timing of operations.

The controller 1102 may be configured to fetch (e.g., obtain, retrieve, receive) instructions from the memory 1104 and determine subsequent instruction(s) to be executed to cause the processor 1100 to support various operations in accordance with examples as described herein. The controller 1102 may be configured to track memory addresses of instructions associated with the memory 1104. The controller 1102 may be configured to decode instructions to determine the operation to be performed and the operands involved. For example, the controller 1102 may be configured to interpret the instruction and determine control signals to be output to other components of the processor 1100 to cause the processor 1100 to support various operations in accordance with examples as described herein. Additionally, or alternatively, the controller 1102 may be configured to manage flow of data within the processor 1100. The controller 1102 may be configured to control transfer of data between registers, ALUs 1106, and other functional units of the processor 1100.

The memory 1104 may include one or more caches (e.g., memory local to or included in the processor 1100 or other memory, such as RAM, ROM, DRAM, SDRAM, SRAM, MRAM, flash memory, etc.). In some implementations, the memory 1104 may reside within or on a processor chipset (e.g., local to the processor 1100). In some other implementations, the memory 1104 may reside external to the processor chipset (e.g., remote to the processor 1100).

The memory 1104 may store computer-readable, computer-executable code including instructions that, when executed by the processor 1100, cause the processor 1100 to perform various functions described herein. The code may be stored in a non-transitory computer-readable medium such as system memory or another type of memory. The controller 1102 and/or the processor 1100 may be configured to execute computer-readable instructions stored in the memory 1104 to cause the processor 1100 to perform various functions. For example, the processor 1100 and/or the controller 1102 may be coupled with or to the memory 1104, the processor 1100, and the controller 1102, and may be configured to perform various functions described herein. In some examples, the processor 1100 may include multiple processors and the memory 1104 may include multiple memories. One or more of the multiple processors may be coupled with one or more of the multiple memories, which may, individually or collectively, be configured to perform various functions herein.

The one or more ALUs 1106 may be configured to support various operations in accordance with examples as described herein. In some implementations, the one or more ALUs 1106 may reside within or on a processor chipset (e.g., the processor 1100). In some other implementations, the one or more ALUs 1106 may reside external to the processor chipset (e.g., the processor 1100). One or more ALUs 1106 may perform one or more computations such as addition, subtraction, multiplication, and division on data. For example, one or more ALUs 1106 may receive input operands and an operation code, which determines an operation to be executed. One or more ALUs 1106 may be configured with a variety of logical and arithmetic circuits, including adders, subtractors, shifters, and logic gates, to process and manipulate the data according to the operation. Additionally, or alternatively, the one or more ALUs 1106 may support logical operations such as AND, OR, exclusive-OR (XOR), not-OR (NOR), and not-AND (NAND), enabling the one or more ALUs 1106 to handle conditional operations, comparisons, and bitwise operations.

The processor 1100 may support wireless communication in accordance with examples as disclosed herein. The processor 1100 may be configured to or operable to support at least one controller (e.g., the controller 1102) coupled with at least one memory (e.g., the memory 1104) and configured to cause the processor to generate a set of multi-resolution features from an input signal; generate a set of resampled multi-resolution features based at least in part on resampling the set of multi-resolution features; and generate a processed output based at least in part on combining the set of resampled multi-resolution features.

Additionally, the processor 1100 may be configured to or operable to support any one or combination of where resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an ECG signal, an EEG, or a PPG signal; a resampling function is implemented to resample the set of multi-resolution features to generate the set of resampled multi-resolution features, and wherein a derivative of the resampling function is expressed in terms of a summation of gradients of the resampling function.

The processor 1100 may support wireless communication in accordance with examples as disclosed herein. The processor 1100 may be configured to or operable to support at least one controller (e.g., the controller 1102) coupled with at least one memory (e.g., the memory 1104) and configured to cause the processor to receive an input signal; generate a set of multi-resolution features based at least in part on processing the input signal; generate a set of resampled multi-resolution features based at least in part on resampling the set of multi-resolution features; and generate an output based at least in part on combining the set of resampled multi-resolution features.

Additionally, the processor 1100 may be configured to or operable to support any one or combination of where resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an ECG signal, an EEG, or a PPG signal; a resampling function is implemented to resample the set of multi-resolution features to generate the set of resampled multi-resolution features, and wherein a derivative of the resampling function is expressed in terms of a summation of gradients of the resampling function.

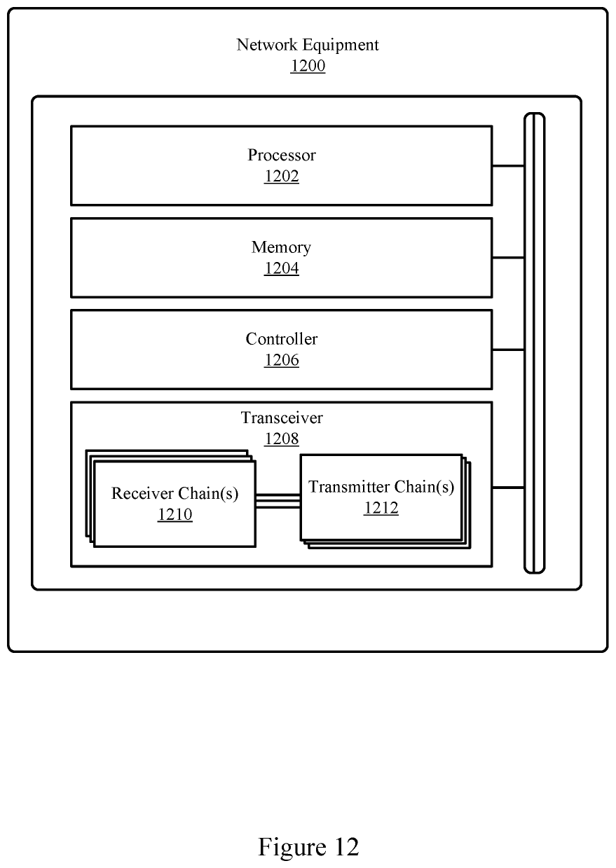

FIG. 12 illustrates an example of an NE 1200 in accordance with aspects of the present disclosure. The NE 1200 may include a processor 1202, a memory 1204, a controller 1206, and a transceiver 1208. The processor 1202, the memory 1204, the controller 1206, or the transceiver 1208, or various combinations thereof or various components thereof may be examples of means for performing various aspects of the present disclosure as described herein. These components may be coupled (e.g., operatively, communicatively, functionally, electronically, electrically) via one or more interfaces.

The processor 1202, the memory 1204, the controller 1206, or the transceiver 1208, or various combinations or components thereof may be implemented in hardware (e.g., circuitry). The hardware may include a processor, a digital signal processor (DSP), an application-specific integrated circuit (ASIC), or other programmable logic device, or any combination thereof configured as or otherwise supporting a means for performing the functions described in the present disclosure.

The processor 1202 may include an intelligent hardware device (e.g., a general-purpose processor, a DSP, a CPU, an ASIC, an FPGA, or any combination thereof). In some implementations, the processor 1202 may be configured to operate the memory 1204. In some other implementations, the memory 1204 may be integrated into the processor 1202. The processor 1202 may be configured to execute computer-readable instructions stored in the memory 1204 to cause the NE 1200 to perform various functions of the present disclosure.

The memory 1204 may include volatile or non-volatile memory. The memory 1204 may store computer-readable, computer-executable code including instructions that, when executed by the processor 1202, cause the NE 1200 to perform various functions described herein. The code may be stored in a non-transitory computer-readable medium such as the memory 1204 or another type of memory. Computer-readable media includes both non-transitory computer storage media and communication media including any medium that facilitates transfer of a computer program from one place to another. A non-transitory storage medium may be any available medium that may be accessed by a general-purpose or special-purpose computer.

In some implementations, the processor 1202 and the memory 1204 coupled with the processor 1202 may be configured to cause the NE 1200 to perform one or more of the functions described herein (e.g., executing, by the processor 1202, instructions stored in the memory 1204). For example, the processor 1202 may support wireless communication at the NE 1200 in accordance with examples as disclosed herein.

The NE 1200 may be configured to or operable to support a means for generating a set of multi-resolution features from an input signal; generating a set of resampled multi-resolution features based at least in part on resampling the set of multi-resolution features; and generating a processed output based at least in part on combining the set of resampled multi-resolution features.

Additionally, the NE 1200 may be configured to or operable to support any one or combination of where resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an ECG signal, an EEG, or a PPG signal; a resampling function is implemented to resample the set of multi-resolution features to generate the set of resampled multi-resolution features, and wherein a derivative of the resampling function is expressed in terms of a summation of gradients of the resampling function.

Additionally, or alternatively, the NE 1200 may support at least one memory (e.g., the memory 1204) and at least one processor (e.g., the processor 1202) coupled with the at least one memory and configured to cause the NE 1200 to generate a set of multi-resolution features from an input signal; generate a set of resampled multi-resolution features based at least in part on resampling the set of multi-resolution features; and generate a processed output based at least in part on combining the set of resampled multi-resolution features.

Additionally, the NE 1200 may be configured to support any one or combination of where resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an ECG signal, an EEG, or a PPG signal; a resampling function is implemented to resample the set of multi-resolution features to generate the set of resampled multi-resolution features, and wherein a derivative of the resampling function is expressed in terms of a summation of gradients of the resampling function.

The NE 1200 may be configured to or operable to support a means for receiving an input signal; generating a set of multi-resolution features based at least in part on processing the input signal; generating a set of resampled multi-resolution features based at least in part on resampling the set of multi-resolution features; and generating an output based at least in part on combining the set of resampled multi-resolution features.

Additionally, the NE 1200 may be configured to or operable to support any one or combination of where resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an ECG signal, an EEG, or a PPG signal; a resampling function is implemented to resample the set of multi-resolution features to generate the set of resampled multi-resolution features, and wherein a derivative of the resampling function is expressed in terms of a summation of gradients of the resampling function.

Additionally, or alternatively, the NE 1200 may support at least one memory (e.g., the memory 1204) and at least one processor (e.g., the processor 1202) coupled with the at least one memory and configured to cause the NE to generate a set of multi-resolution features from an input signal; generate a set of resampled multi-resolution features based at least in part on resampling the set of multi-resolution features; and generate a processed output based at least in part on combining the set of resampled multi-resolution features.

Additionally, the NE 1200 may be configured to support any one or combination of where resampling the set of multi-resolution features includes: up-sampling the set of multi-resolution features to generate the set of resampled multi-resolution features; the set of multi-resolution features are up-sampled at multiple different resolutions to generate the set of resampled multi-resolution features; resampling the set of multi-resolution features includes: down-sampling the set of multi-resolution features to generate the set of resampled multi-resolution features; the set of multi-resolution features are down-sampled at multiple different resolutions to generate the set of resampled multi-resolution features; the input signal includes one or more of an audio signal, a video signal, or an image signal; the input signal includes a human-based physiological signal; the human-based physiological signal includes at least one of an ECG signal, an EEG, or a PPG signal; a resampling function is implemented to resample the set of multi-resolution features to generate the set of resampled multi-resolution features, and wherein a derivative of the resampling function is expressed in terms of a summation of gradients of the resampling function.

The controller 1206 may manage input and output signals for the NE 1200. The controller 1206 may also manage peripherals not integrated into the NE 1200. In some implementations, the controller 1206 may utilize an operating system such as iOS®, ANDROID®, WINDOWS®, or other operating systems. In some implementations, the controller 1206 may be implemented as part of the processor 1202.

In some implementations, the NE 1200 may include at least one transceiver 1208. In some other implementations, the NE 1200 may have more than one transceiver 1208. The transceiver 1208 may represent a wireless transceiver. The transceiver 1208 may include one or more receiver chains 1210, one or more transmitter chains 1212, or a combination thereof.

A receiver chain 1210 may be configured to receive signals (e.g., control information, data, packets) over a wireless medium. For example, the receiver chain 1210 may include one or more antennas to receive a signal over the air or wireless medium. The receiver chain 1210 may include at least one amplifier (e.g., a low-noise amplifier (LNA)) configured to amplify the received signal. The receiver chain 1210 may include at least one demodulator configured to demodulate the received signal and obtain the transmitted data by reversing the modulation technique applied during transmission of the signal. The receiver chain 1210 may include at least one decoder for decoding the demodulated signal to receive the transmitted data.

A transmitter chain 1212 may be configured to generate and transmit signals (e.g., control information, data, packets). The transmitter chain 1212 may include at least one modulator for modulating data onto a carrier signal, preparing the signal for transmission over a wireless medium. The at least one modulator may be configured to support one or more techniques such as amplitude modulation (AM), frequency modulation (FM), or digital modulation schemes like phase-shift keying (PSK) or quadrature amplitude modulation (QAM). The transmitter chain 1212 may also include at least one power amplifier configured to amplify the modulated signal to an appropriate power level suitable for transmission over the wireless medium. The transmitter chain 1212 may also include one or more antennas for transmitting the amplified signal into the air or wireless medium.

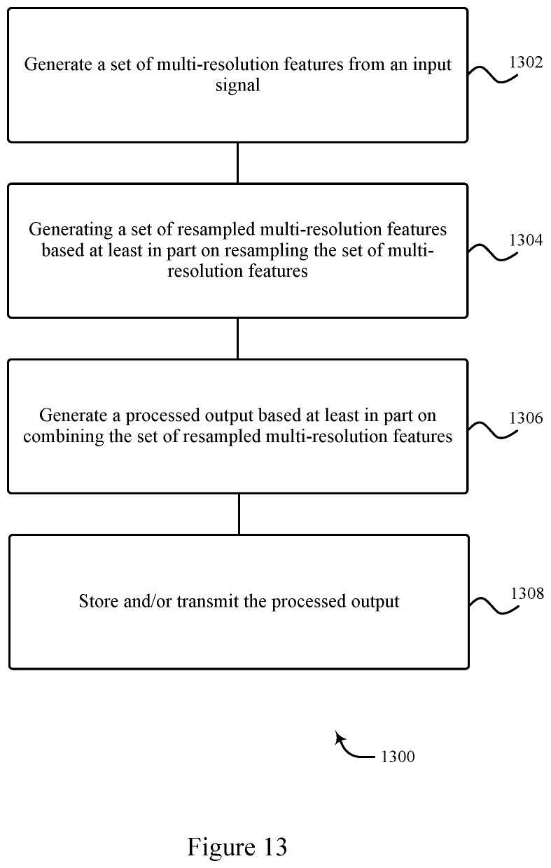

FIG. 13 illustrates a flowchart of a method 1300 in accordance with aspects of the present disclosure. The operations of the method may be implemented by a UE and/or an NE as described herein. In some implementations, the UE and/or the NE may execute a set of instructions to control the functional elements of the UE and/or the NE to perform the described functions. It should be noted that the method described herein describes a possible implementation, and that the operations and the steps may be rearranged or otherwise modified and that other implementations are possible.

At 1302, the method may include generating a set of multi-resolution features from an input signal. The operations of 1302 may be performed in accordance with examples as described herein. In some implementations, aspects of the operations of 1302 may be performed by a UE as described with reference to FIG. 10 and/or an NE as described with reference to FIG. 12.

At 1304, the method may include generating a set of resampled multi-resolution features based at least in part on resampling the set of multi-resolution features. The operations of 1304 may be performed in accordance with examples as described herein. In some implementations, aspects of the operations of 1304 may be performed by a UE as described with reference to FIG. 10 and/or an NE as described with reference to FIG. 12.

At 1306, the method may include generating a processed output based at least in part on combining the set of resampled multi-resolution features. The operations of 1306 may be performed in accordance with examples as described herein. In some implementations, aspects of the operations of 1306 may be performed by a UE as described with reference to FIG. 10 and/or an NE as described with reference to FIG. 12.

At 1308, the method may include storing and/or transmitting the processed output.

The operations of 1308 may be performed in accordance with examples as described herein. In some implementations, aspects of the operations of 1308 may be performed by a UE as described with reference to FIG. 10 and/or an NE as described with reference to FIG. 12.

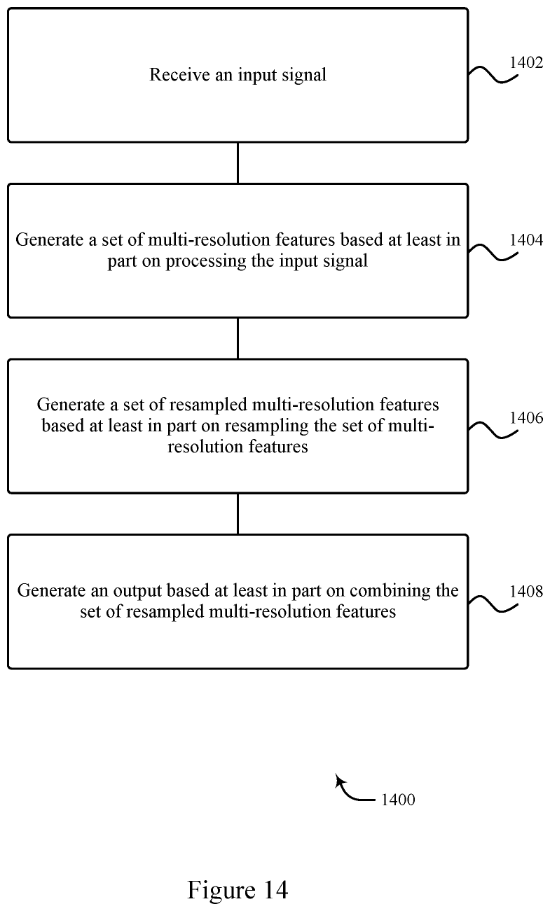

FIG. 14 illustrates a flowchart of a method 1400 in accordance with aspects of the present disclosure. The operations of the method may be implemented by a UE and/or an NE as described herein. In some implementations, the UE and/or the NE may execute a set of instructions to control the functional elements of the UE and/or the NE to perform the described functions. It should be noted that the method described herein describes a possible implementation, and that the operations and the steps may be rearranged or otherwise modified and that other implementations are possible.

At 1402, the method may include receiving an input signal. The operations of 1402 may be performed in accordance with examples as described herein. In some implementations, aspects of the operations of 1402 may be performed by a UE as described with reference to FIG. 10 and/or an NE as described with reference to FIG. 12.

At 1404, the method may include generating a set of multi-resolution features based at least in part on processing the input signal. The operations of 1404 may be performed in accordance with examples as described herein. In some implementations, aspects of the operations of 1404 may be performed by a UE as described with reference to FIG. 10 and/or an NE as described with reference to FIG. 12.

At 1406, the method may include generating a set of resampled multi-resolution features based at least in part on resampling the set of multi-resolution features. The operations of 1406 may be performed in accordance with examples as described herein. In some implementations, aspects of the operations of 1406 may be performed by a UE as described with reference to FIG. 10 and/or an NE as described with reference to FIG. 12.

At 1408, the method may include generating an output based at least in part on combining the set of resampled multi-resolution features. The operations of 1408 may be performed in accordance with examples as described herein. In some implementations, aspects of the operations of 1408 may be performed by a UE as described with reference to FIG. 10 and/or an NE as described with reference to FIG. 12.

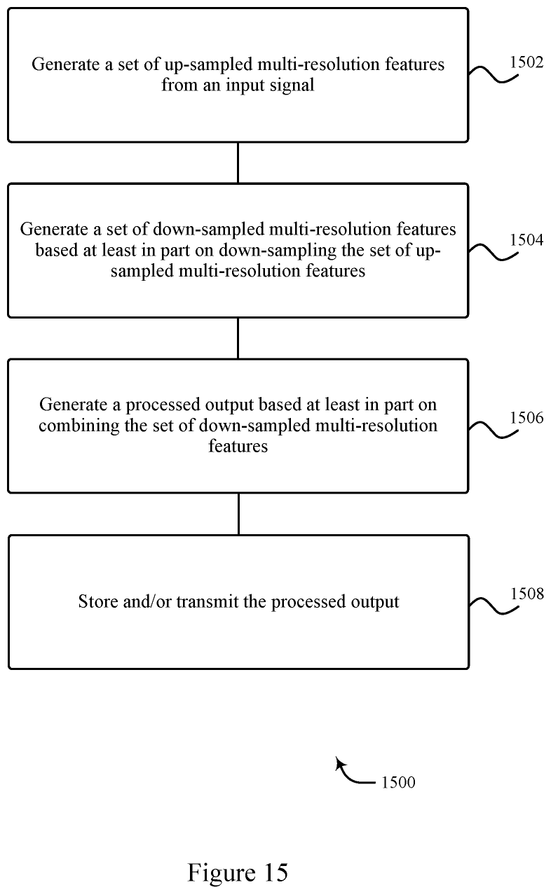

FIG. 15 illustrates a flowchart of a method 1500 in accordance with aspects of the present disclosure. The operations of the method may be implemented by a UE and/or an NE as described herein. In some implementations, the UE and/or the NE may execute a set of instructions to control the functional elements of the UE and/or the NE to perform the described functions. It should be noted that the method described herein describes a possible implementation, and that the operations and the steps may be rearranged or otherwise modified and that other implementations are possible.

At 1502, the method may include generating a set of up-sampled multi-resolution features from an input signal. The operations of 1502 may be performed in accordance with examples as described herein. In some implementations, aspects of the operations of 1502 may be performed by a UE as described with reference to FIG. 10 and/or an NE as described with reference to FIG. 12.

At 1504, the method may include generating a set of down-sampled multi-resolution features based at least in part on down-sampling the set of up-sampled multi-resolution features. The operations of 1504 may be performed in accordance with examples as described herein. In some implementations, aspects of the operations of 1504 may be performed by a UE as described with reference to FIG. 10 and/or an NE as described with reference to FIG. 12.

At 1506, the method may include generating a processed output based at least in part on combining the set of down-sampled multi-resolution features. The operations of 1506 may be performed in accordance with examples as described herein. In some implementations, aspects of the operations of 1506 may be performed by a UE as described with reference to FIG. 10 and/or an NE as described with reference to FIG. 12.

At 1508, the method may include storing and/or transmitting the processed output. The operations of 1508 may be performed in accordance with examples as described herein. In some implementations, aspects of the operations of 1508 may be performed by a UE as described with reference to FIG. 10 and/or an NE as described with reference to FIG. 12.

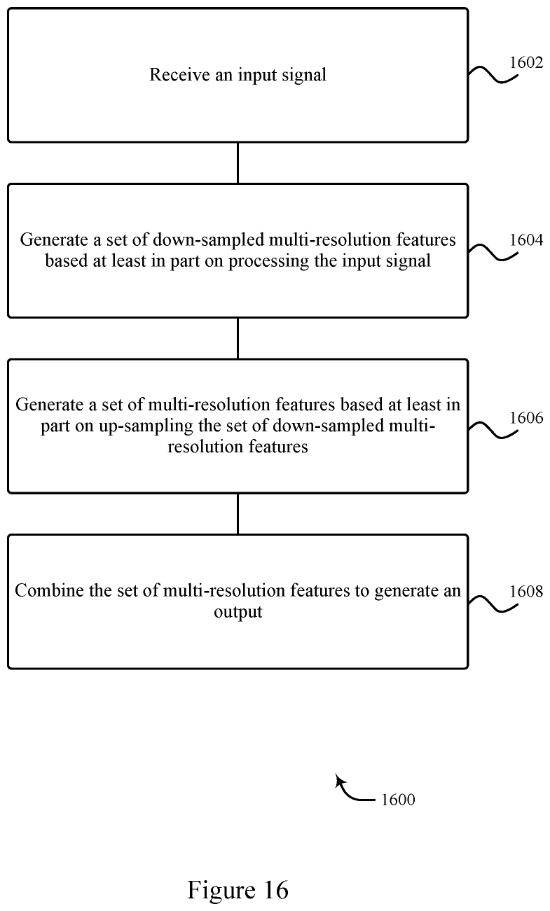

FIG. 16 illustrates a flowchart of a method 1600 in accordance with aspects of the present disclosure. The operations of the method may be implemented by a UE and/or an NE as described herein. In some implementations, the UE and/or the NE may execute a set of instructions to control the functional elements of the UE and/or the NE to perform the described functions. It should be noted that the method described herein describes a possible implementation, and that the operations and the steps may be rearranged or otherwise modified and that other implementations are possible.

At 1602, the method may include receiving an input signal. The operations of 1602 may be performed in accordance with examples as described herein. In some implementations, aspects of the operations of 1602 may be performed by a UE as described with reference to FIG. 10 and/or an NE as described with reference to FIG. 12.

At 1604, the method may include generating a set of down-sampled multi-resolution features based at least in part on processing the input signal. The operations of 1604 may be performed in accordance with examples as described herein. In some implementations, aspects of the operations of 1604 may be performed by a UE as described with reference to FIG. 10 and/or an NE as described with reference to FIG. 12.

At 1606, the method may include generating a set of multi-resolution features based at least in part on up-sampling the set of down-sampled multi-resolution features. The operations of 1606 may be performed in accordance with examples as described herein. In some implementations, aspects of the operations of 1606 may be performed by a UE as described with reference to FIG. 10 and/or an NE as described with reference to FIG. 12.

At 1608, the method may include combining the set of multi-resolution features to generate an output. The operations of 1608 may be performed in accordance with examples as described herein. In some implementations, aspects of the operations of 1608 may be performed by a UE as described with reference to FIG. 10 and/or an NE as described with reference to FIG. 12.

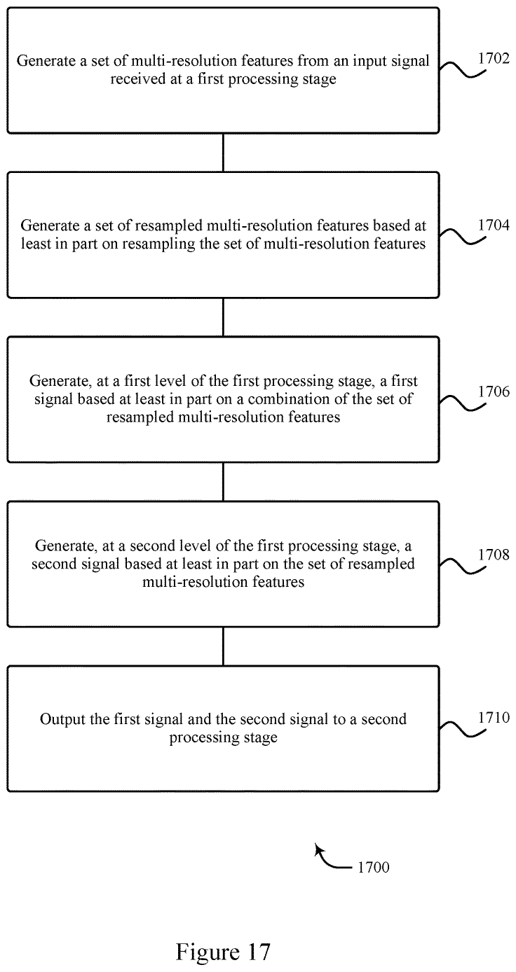

FIG. 17 illustrates a flowchart of a method 1700 in accordance with aspects of the present disclosure. The operations of the method may be implemented by a UE and/or an NE as described herein. In some implementations, the UE and/or the NE may execute a set of instructions to control the functional elements of the UE and/or the NE to perform the described functions. It should be noted that the method described herein describes a possible implementation, and that the operations and the steps may be rearranged or otherwise modified and that other implementations are possible.

At 1702, the method may include generating a set of multi-resolution features from an input signal received at a first processing stage. The operations of 1702 may be performed in accordance with examples as described herein. In some implementations, aspects of the operations of 1702 may be performed by a UE as described with reference to FIG. 10 and/or an NE as described with reference to FIG. 12.

At 1704, the method may include generating a set of resampled multi-resolution features based at least in part on resampling the set of multi-resolution features. The operations of 1704 may be performed in accordance with examples as described herein. In some implementations, aspects of the operations of 1704 may be performed by a UE as described with reference to FIG. 10 and/or an NE as described with reference to FIG. 12.

At 1706, the method may include generating, at a first level of the first processing stage, a first signal based at least in part on a combination of the set of resampled multi-resolution features. The operations of 1706 may be performed in accordance with examples as described herein. In some implementations, aspects of the operations of 1706 may be performed by a UE as described with reference to FIG. 10 and/or an NE as described with reference to FIG. 12.

At 1708, the method may include generating, at a second level of the first processing stage, a second signal based at least in part on the set of resampled multi-resolution features. The operations of 1708 may be performed in accordance with examples as described herein. In some implementations, aspects of the operations of 1708 may be performed by a UE as described with reference to FIG. 10 and/or an NE as described with reference to FIG. 12.

At 1710, the method may include outputting the first signal and the second signal to a second processing stage. The operations of 1710 may be performed in accordance with examples as described herein. In some implementations, aspects of the operations of 1710 may be performed by a UE as described with reference to FIG. 10 and/or an NE as described with reference to FIG. 12.

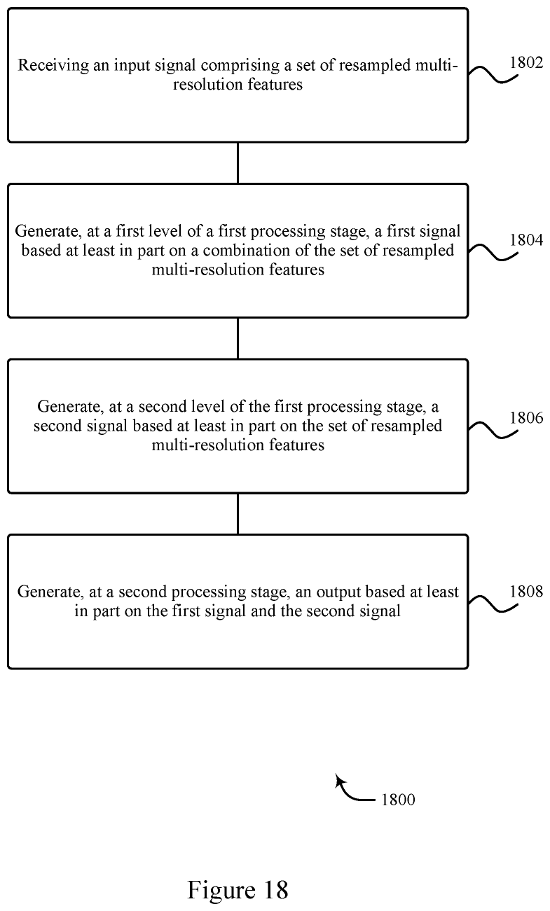

FIG. 18 illustrates a flowchart of a method 1800 in accordance with aspects of the present disclosure. The operations of the method may be implemented by a UE and/or an NE as described herein. In some implementations, the UE and/or the NE may execute a set of instructions to control the functional elements of the UE and/or the NE to perform the described functions. It should be noted that the method described herein describes a possible implementation, and that the operations and the steps may be rearranged or otherwise modified and that other implementations are possible.

At 1802, the method may include receiving an input signal comprising a set of resampled multi-resolution features. The operations of 1802 may be performed in accordance with examples as described herein. In some implementations, aspects of the operations of 1802 may be performed by a UE as described with reference to FIG. 10 and/or an NE as described with reference to FIG. 12.

At 1804, the method may include generating, at a first level of a first processing stage, a first signal based at least in part on a combination of the set of resampled multi-resolution features. The operations of 1804 may be performed in accordance with examples as described herein. In some implementations, aspects of the operations of 1804 may be performed by a UE as described with reference to FIG. 10 and/or an NE as described with reference to FIG. 12.

At 1806, the method may include generating, at a second level of the first processing stage, a second signal based at least in part on the set of resampled multi-resolution features. The operations of 1806 may be performed in accordance with examples as described herein. In some implementations, aspects of the operations of 1806 may be performed by a UE as described with reference to FIG. 10 and/or an NE as described with reference to FIG. 12.

At 1808, the method may include generating, at a second processing stage, an output based at least in part on the first signal and the second signal. The operations of 1808 may be performed in accordance with examples as described herein. In some implementations, aspects of the operations of 1808 may be performed by a UE as described with reference to FIG. 10 and/or an NE as described with reference to FIG. 12.

The description herein is provided to enable a person having ordinary skill in the art to make or use the disclosure. Various modifications to the disclosure will be apparent to a person having ordinary skill in the art, and the generic principles defined herein may be applied to other variations without departing from the scope of the disclosure. Thus, the disclosure is not limited to the examples and designs described herein but is to be accorded the broadest scope consistent with the principles and novel features disclosed herein.

What is claimed is:

1. A first apparatus for wireless communication, comprising:
at least one memory; and
at least one processor coupled with the at least one memory and operable to cause the first apparatus to:
generate a set of multi-resolution features from an input signal;
generate a set of resampled multi-resolution features based at least in part on resampling the set of multi-resolution features; and
generate a processed output based at least in part on combining the set of resampled multi-resolution features.

2. The first apparatus of claim 1, wherein resampling the set of multi-resolution features comprises:
up-sampling the set of multi-resolution features to generate the set of resampled multi-resolution features.

3. The first apparatus of claim 2, wherein the set of multi-resolution features are up-sampled at multiple different resolutions to generate the set of resampled multi-resolution features.

4. The first apparatus of claim 1, wherein resampling the set of multi-resolution features comprises:
down-sampling the set of multi-resolution features to generate the set of resampled multi-resolution features.

5. The first apparatus of claim 4, wherein the set of multi-resolution features are down-sampled at multiple different resolutions to generate the set of resampled multi-resolution features.

6. The first apparatus of claim 1, wherein the input signal comprises one or more of an audio signal, a video signal, or an image signal.

7. The first apparatus of claim 1, wherein the input signal comprises a human-based physiological signal.

8. The first apparatus of claim 7, wherein the human-based physiological signal comprises at least one of an electrocardiogram (ECG) signal, an electroencephalogram (EEG) signal, or a photoplethysmography (PPG) signal.

9. The first apparatus of claim 1, wherein a resampling function is implemented to resample the set of multi-resolution features to generate the set of resampled multi-resolution features, and wherein a derivative of the resampling function is expressed in terms of a summation of gradients of the resampling function.

10. A second apparatus for wireless communication, comprising:
at least one memory; and
at least one processor coupled with the at least one memory and operable to cause the second apparatus to:
receive an input signal;
generate a set of multi-resolution features based at least in part on processing the input signal;
generate a set of resampled multi-resolution features based at least in part on resampling the set of multi-resolution features; and
generate an output based at least in part on combining the set of resampled multi-resolution features.

11. The second apparatus of claim 10, wherein resampling the set of multi-resolution features comprises:
up-sampling the set of multi-resolution features to generate the set of resampled multi-resolution features.

12. The second apparatus of claim 11, wherein the set of multi-resolution features are up-sampled at multiple different resolutions to generate the set of resampled multi-resolution features.

13. The second apparatus of claim 10, wherein resampling the set of multi-resolution features comprises:
down-sampling the set of multi-resolution features to generate the set of resampled multi-resolution features.

14. The second apparatus of claim 13, wherein the set of multi-resolution features are down-sampled at multiple different resolutions to generate the set of resampled multi-resolution features.

15. The second apparatus of claim 10, wherein the input signal comprises one or more of an audio signal, a video signal, or an image signal.

16. The second apparatus of claim 10, wherein the input signal comprises a human-based physiological signal.

17. The second apparatus of claim 16, wherein the human-based physiological signal comprises at least one of an electrocardiogram (ECG) signal, an electroencephalogram (EEG) signal, or a photoplethysmography (PPG) signal.

18. The second apparatus of claim 10, wherein a resampling function is implemented to resample the set of multi-resolution features to generate the set of resampled multi-resolution features, and wherein a derivative of the resampling function is expressed in terms of a summation of gradients of the resampling function.

19. A method performed by a first apparatus, the method comprising:
generating a set of multi-resolution features from an input signal;
generating a set of resampled multi-resolution features based at least in part on resampling the set of multi-resolution features; and
generating a processed output based at least in part on combining the set of resampled multi-resolution features.

20. A method performed by a second apparatus, the method comprising:
receiving an input signal;
generating a set of multi-resolution features based at least in part on processing the input signal to;
generating a set of resampled multi-resolution features based at least in part on resampling the set of multi-resolution features; and
generating an output based at least in part on combining the set of resampled multi-resolution features.
